(19) United States
(12) Patent Application Publication
(10) Pub. No.: US 2006/0058816 A1
(43) Pub. Date: Mar. 16, 2006

(54) Disposable device for multi-ligation of bleeding esophageal varices

(76) Inventor: Ali Mohamed Ali Hassanien, Roshdy (EG)

Correspondence Address: WELSH & KATZ, LTD, 120 S RIVERSIDE PLAZA, 22ND FLOOR, CHICAGO, IL, 60606, US

(21) Appl. No.: 11/269,210

(22) Filed: Nov. 8, 2005

Related U.S. Application Data
(63) Continuation-in-part of application No. PCT/EG03/00014, filed on Dec. 27, 2003.

(30) Foreign Application Priority Data
Dec. 28, 2002 (EG) 2002121401

(51) Int. Cl. A61B 17/10

(52) U.S. Cl. 606/139

(57) ABSTRACT

A disposable device for ligation of bleeding Esophageal varices comprising two main parts including a three arm part carrying a plurality of rubber bands and a pressure providing part. The pressure providing part includes a pressure cylinder and a spring is provided between the cylinder and the three arm threaded or serrated part and a pressure line is connected to the cylinder for applying pressure against the three arm threaded or serrated part in order to release a rubber band from the three arm threaded or serrated part.

### CROSS REFERENCE TO RELATED APPLICATIONS

This application is a continuation in part of PCT application number PCT/EG2003/000014, filed Dec. 23, 2003 which claims priority from Egyptian Patent application number 2002121401, filed Dec. 28, 2002.

### BACKGROUND OF THE INVENTION

1. Field of the Invention

This invention relates to disposable device for ligation of bleeding Esophageal varices. In particular the invention directed to a new disposable device for the ligation of bleeding Esophageal varices characterized in that it has a new mechanism and can be used for multi-ligation of the Esophageal varices.

2. Description of the Related Art

Bleeding Esophageal varices is a severe complication of various liver diseases as a result of the high venous pressure on the esophageal region. The main diseases that lead to the Bleeding Esophageal varices includes:
1—Liver parasites such as Bilharzias
2—Various kinds of Hepatitis.
3—Liver fibrosis of various origin (e.g. from drugs, alcohol liver toxicity etc.)

Due to the high venous pressure on the esophageal region, hemorrhoids start to develop and immediately bleeding occurs. Where bleeding occurs, there is a need to achieve hemostasis in bleeding varices and minimize recurrence of this bleeding which may lead eventually to death. Presently, these Bleeding Esophageal varices are treated by any of the following methods:

1—by pass surgery to redirect the main veins and reduce pressure in the esophageal region.

2—Injection sclerotherapy using endoscope.

3—band ligation utilizes an endoscope.

Currently, therapeutic techniques includes injection sclerotherapy and band ligation. These techniques are utilized for the immediate and long term control of hemorrhaging associated with esophageal varices.

In sclerotherapy, a solution, such as sodium morrhuate or ethanolamine is injected submucosally, utilizing endoscope, into the tissue around the varicose vein in the esophagus to cause inflammation and scarring to close off the vein and reduce the likelihood of bleeding.

Disadvantages of these methods include the following:

1—It needs experienced surgeon to handle the injection, to avoid injected the material in non-target regions.

2—Some of the bleeding veins can not be handled by this process.

3—repeated injection may create ulcerations or fibrosis which can lead to esophageal strictures.

Ligation is effective method to treat this case using endoscope. The esophageal varices are ensnared with elastic bands to eradicate the varices. Presently, there is a device for effecting this kind of ligation, this device is working through a mechanism that includes pulling the varices to the unit and release the rubber band by pulling a string.

The use of this device is very critical since it may cause a damage to the endoscope itself and releasing of one rubber band only depend on the pulling forces if it too strong two or more rubber bands may be released.

Other ligation devices are known but the can be used to ligate only one varices at a time and then has to be taken out and reloaded which is inconvenient for both of the patient and the surgeon.

In U.S. Pat. No. 5,707,355, Zimmon; a method and apparatus for treating esophageal varices and mucosal neoplasm. In this method, a balloon tamponade device is utilized to deliver a dressing containing a sclerosant agent to bleeding esophageal.

U.S. Pat. No. 5,400,770, Nakao et at, disclosed a surgical device for use with endoscope to perform gastroesophageal hemostasis operations. The device comprises an insertion member, inflatable balloon and attachment component.

U.S. Pat. No. 6,139,527, Laufer et at, disclose a method and apparatus for treating hemorrhoids wherein a catheter delivers an electrode within a vein for a minimally invasive treatment of hemorroids using RF energy.

Yet a need exists in the art to treat the bleeding veins which give rise to esophageal varices and reduce venous pressure on the esophageal region from the portal vein system without the attendant risks of invasive surgery. Further need exists to provide a less invasive procedure which can treat multiple venous sites quickly and easily. There is still a need to achieve hemostasis in bleeding varices and minimize recurrence of bleeding.

### BRIEF SUMMARY OF THE INVENTION

The present invention relates to a device for treating bleeding esophagus. The device comprises three cylindrical parts. The first cylindrical part has toothed, serrated or threaded rows with equal dimensions and arranged parallel to each other along its cylindrical surface. The second part represents part of a cylinder with three toothed, serrated or threaded arms situated vertically on it, i.e., extending radially outwardly from its cylindrical surface, and being similar in shape and dimensions to the toothes, serrated or threaded rows. The third part comprise two cylinders inside each other and connected together on one side and free on the other side to be compatible with the other parts. In a preferred embodiment the second part and third part are joined together to form one part and a spring between the first and second parts is located to push the toothed, serrated or threaded arms axially outwardly.

According to the teachings of the present invention there is provided a disposable device for ligation of bleeding Esophageal varices comprising two main parts including a threaded arms part and a pressure providing part and the pressure providing part includes a pressure cylinder and a spring is provided on a side wall of the pressure cylinder in connection with the bottom of the threaded arms to push the threaded arms upon using pressure in order to release a rubber band.

### BRIEF DESCRIPTION OF THE SEVERAL VIEWS OF THE DRAWINGS

FIG. 1 is schematic perspective view showing the device of the invention.

FIG. 2. schematic perspective view showing the threads.

FIG. 3 cross sectional view showing the components of the cylinder.

FIG. 4. Cross sectional taken along the line A-A of FIG. 1.

FIG. 5. schematic end view of the cylinder showing the position of the three arms.

FIG. 6. Cross sectional view showing the position of the pressure tube.

FIG. 7 schematic perspective view showing an external additional part used for connection.

FIG. 8 cross sectional view of the part shown in FIG. 7.

FIG. 9 Cross sectional view showing preferred embodiment of the device of the invention.

FIG. 10 Cross sectional taken along the line A-A of FIG. 9.

### DETAILED DESCRIPTION OF THE PREFERRED EMBODIMENTS

The present invention relates to device for multi-ligation of bleeding Esophageal varices. The new device will be better explained with reference to the accompanying drawings.

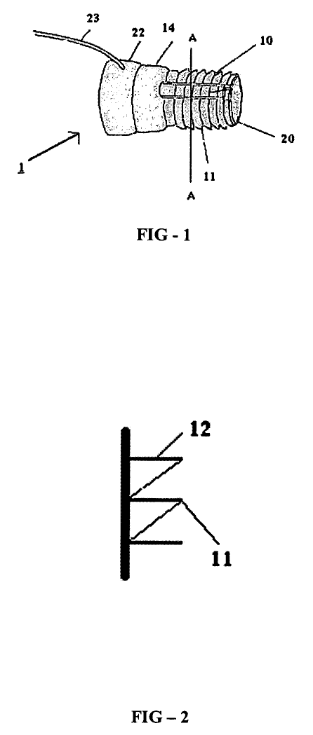

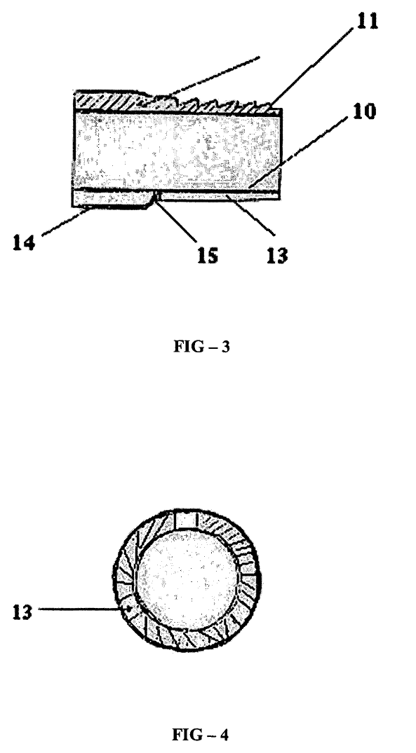

As illustrated in FIG. 1, the device (1) consists of three main parts. The first part is a cylinder (10) characterized in that the first two thirds of this cylinder is threaded or serrated. Threads or serrations (11) are circumscribed and as shown in FIG. 2 it has a right angled triangle cross section. Wherein the altitude (12) of the triangle upwardly positioned. Distances and dimensions of all threads are identical. Cutting these threads longitudinal, three identical equally spaced grooves in the form of passageways allowing tongues to pass through as better shown in FIG. 4 which is cross section taken along line A-A of FIG. 1.

The last third of the cylinder (10) is not threaded and covered by another cylinder (14). Both cylinder having equal length, however, cylinder (14) is bigger in diameter. Cylinders 10 and 14 are integrated completely except for the position of the three grooves (13). Fig three represent a cross section of the two unified cylinders (10) and (14).

The second part is shown in FIG. 5 and consists of a base cylinder (16) having three arm (17). The arm (17) having threads (18) which are identical in all respects with threads (11) of the first part of the device. And having unthreaded part (21) connecting the threaded arms with the base cylinder. Dimensions of the arm (17) adopted to fit in the grooves (13) easily and to form altogether homogeneous unit with the first part. The base cylinder (16) is introduced in the space created by the last third of cylinder (10) and cylinder (14) so that arms can be inserted in the grooves and base cylinder moves upwardly in the space and the arm (17) emerge and appeared on the surface of the threaded part of cylinder (1) and integrated therewith so that when arm (17) reach the top part of the device (20) the threads of the cylinder (10) are completed by the threaded arm (17). Within the unthreaded part on the arm (17), there is a special spring (19) which is used to trigger the arms upwardly and return it back after triggering.

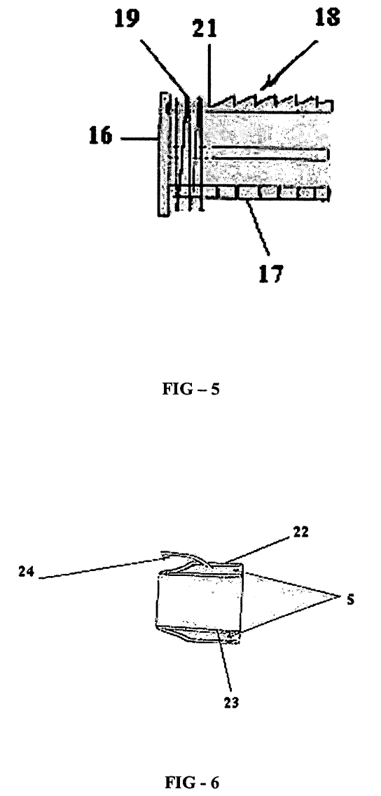

FIG. 6 represents the third part of the device which consists of two uni-center cylinders (22) and (23) one having bigger diameter (23) than the other which create a space (5) in between. There is a micro tube (24) connected to the space from one side and connected to a pressure source (not shown in the drawing) from the other side.

As mentioned above the device can be easily used to ligate bleeding Esophageal varices using rubber bands loaded inside the device. The number of bands loaded into device can vary from 4 up to 10, preferably from 4 to 8 and most preferable 6 rubber bands.

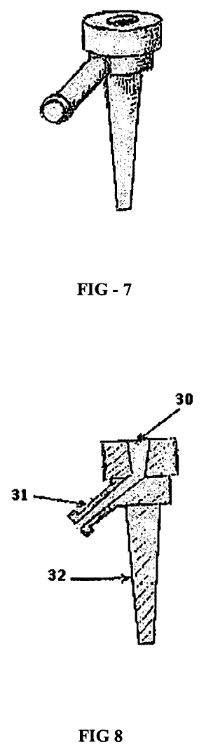

In another aspect of the invention, a new external part has been added to the device shown in FIG. 7 to provide pressure required for release of the ligation band. The part is shown in FIG. 7 which is a schematic drawing of the part in general. FIG. 8 is a cross section of the part shown in FIG. 7 where there is shown a port 30, a conduit 31 and a cone shaped connecting tube 32.

To simplify use of the unit, a normal syringe is used to provide the required pressure, the syringe is connected and fixed in port 30. A tube 23 is extended from the device to be connected with the conduit 31 of the external part. The device is normally connected to an endoscope. In the present embodiment, an endoscope is connected to the cone shaped connecting tube 32.

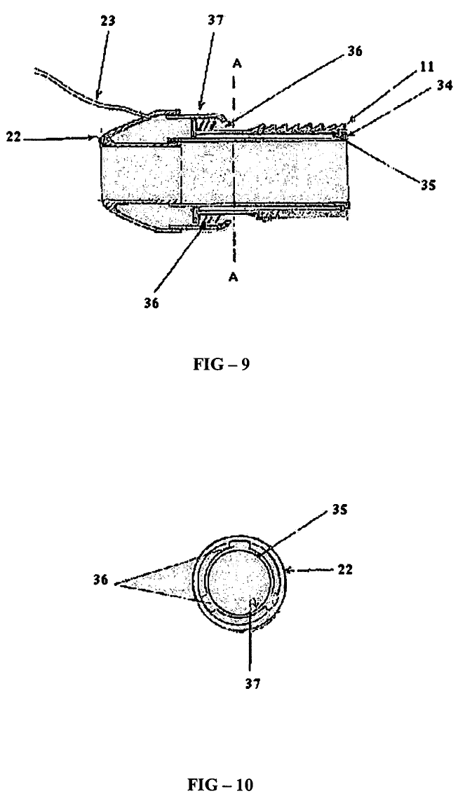

In one preferred embodiment of the invention, there is a device for ligation of bleeding Esophageal varices shown in FIG. 9. The device is almost identical with the device shown in FIG. 1 except for some slight variations. Device 40 comprises two major parts where part 2 and 3 shown in FIG. 1 have been merged to form one part. In this device, spring 36 has been inserted in a gap between the outer wall 37 and a three arm threaded or serrated part 11. Part 22 is an empty enclosed space connected through pipe 23 to pressure source. When pressure is applied this pressure result in pushing the threaded or serrated three arm part to release ligation bands. There is an empty space 35 between the threaded or serrated three arm part 11 and the inner wall of the tube which allows the arms to move freely. FIG. 10 is a cross section taken along the line A-A in FIG. 9 and shows the outer wall of the cylinder 22, the springs 36 and the inner wall of the serrated or threaded three arm part 35.

The practice of the invention is illustrated in the following non-limiting example.

Exemplary Procedure:

The device in this example is loaded with 6 rubber bands so that the first rubber band is immediately in front of the three arms.

The unit is connected with the endoscope and inserted into the patient from the mouth up till Esophagea. The surgeon determine the bleeding varices which is withdrawn by the endoscope. Using a small air pressure (e.g. air pressure created by a syringe) the surgeon trigger the first rubber band upon using the pressure. Once pressure is used the three arm serrated or threaded part 11 pushes the first rubber band to ligate the varice already withdrawn and in the mean time the spring return the arms back to the original position to reposition a new rubber band to replace the one already released.

What is claimed is:

1. Disposable device for ligation of bleeding Esophageal varices characterized by comprising three main cylindrical parts.

2. The disposable device for ligation of bleeding Esophageal varices according to claim 1 wherein the first part is a cylinder characterized in that the first two thirds of this cylinder is threaded or serrated, the last third of the cylinder is not threaded and covered by another cylinder, both cylinders having equal length, however, one of them is bigger in diameter and the cylinders are integrated completely except for the position of three grooves.

3. The disposable device for ligation of bleeding Esophageal varices according to claim 2 wherein threads are circumscribed or are serrations and have a right angled triangle cross section wherein the altitude of the triangle is upwardly positioned and the distances of and dimensions of all threads are identical.

4. The disposable device for ligation of bleeding Esophageal varices according to claim 2 wherein threads are cut longitudinal by three identical equally spaced grooves in the form of passageways allowing tongues to pass through.

5. The disposable device for ligation of bleeding Esophageal varices according to claim 1 wherein the second part comprises a base cylinder having three arms emerging therefrom, the arms having threads or serrations which are identical in all respects with threads of the first part of the device, and having an unthreaded part connecting the threaded arms with the base cylinder, and within the unthreaded part of the arm, there is a special spring which is used to trigger the arms upwardly and return it back after triggering.

6. The disposable device for ligation of bleeding Esophageal varices according to claim 5 wherein threads or serrations are identical in all respects with threads or serrations of the first part of the device.

7. The disposable device for ligation of bleeding Esophageal varices according to claim 1 the third part of the device consists of two uni-center cylinders one having bigger diameter than the other creating a space in between, there being a micro long tube connected to the said space from one side and connected to pressure source from the other side.

8. The disposable device for ligation of bleeding Esophageal varices according to claim 1 wherein the device is loaded with a number of rubber bands amounting to 6 or more.

9. The disposable device for ligation of bleeding Esophageal varices according to claim 8 characterized in that one rubber band only can be released each time the device is triggered.

10. The disposable device for ligation of bleeding Esophageal varices according to claim 9 wherein the mechanism of releasing the rubber band depend on a slight pressure created either by air or distilled water.

11. The disposable device for ligation of bleeding Esophageal varices according to claim 10 wherein the pressure source used is a medical syringe.

12. The disposable device for ligation of bleeding Esophageal varices according to claim 10 wherein an external part is used to connect with a pressure pipe.

13. The disposable device for ligation of bleeding Esophageal varices according to claim 10 including a pressure source and an endoscope.

14. The disposable device for ligation of bleeding Esophageal varices according to claim 13 wherein the pressure source is a simple medical syringe.

15. A disposable device for ligation of bleeding Esophageal varices comprising two main parts including a three arm threaded or serrated part carrying a plurality of rubber bands and a pressure providing part.

16. The disposable device for ligation of bleeding Esophageal varices according to claim 15 wherein the pressure providing part includes a pressure cylinder and a spring is provided between the cylinder and the three arm threaded or serrated part and a pressure line is connected to the cylinder for applying pressure against the three arm threaded or serrated part in order to release a rubber band from the three arm threaded or serrated part.
